(19) United States
(12) Patent Application Publication
(10) Pub. No.: US 2025/0204550 A1
(43) Pub. Date: Jun. 26, 2025

(54) MICROBIAL CELL EXTRACT

(71) Applicant: FUMI Holding B.V., Wageningen (NL)

(72) Inventors: Edgar Suarez GARCIA, Wageningen (NL); Corjan VANDENBERG, Wageningen (NL)

(21) Appl. No.: 18/848,357

(22) PCT Filed: Mar. 17, 2023
(86) PCT No.: PCT/EP2023/056902

§ 371 Date: Sep. 18, 2024

(30) Foreign Application Priority Data
Mar. 18, 2022 (NL) 2031323

(51) Int. Cl. A23J 1/18; A23J 3/18; A23J 3/22; A23J 3/26; C12N 1/06

(52) U.S. Cl. CPC A23J 1/18; A23J 3/18; A23J 3/227; A23J 3/26; C12N 1/066

(57) ABSTRACT

A process for preparing a microbial cell extract for food use is described, comprising mechanically disrupting cells (eg, yeast cell), and separating the disrupted cells into fractions. The fractions can be recombined and further processed to provide texture or other qualities which resemble meat.

### TECHNICAL FIELD

The present invention relates to a method for producing a microbial cell extract. The invention further relates to a microbial cell extract obtained by or obtainable by said method, the processing of said microbial extract and compositions including said microbial cell extract. The invention further relates to the use of compositions comprising said microbial cell extract in various applications including food preparations.

### BACKGROUND

The rapid increase in the world population has caused a substantial rise in the demand for food. To cope with this demand, agricultural activities have seen a massive expansion in order to provide sufficient food products or crops to sustain large scale animal farming. Intense agricultural activities and animal farming come with several sustainability issues such as overexploitation of water and land resources, greenhouse emissions and deforestation. Furthermore, ethical and health concerns are also present, due to animal welfare issues and high usage of antibiotics and hormones in conventional farming practices. In this regard, a reduction in the consumption of meat, the replacement of animal products by vegetables and crops, and the development of structured products such as tofu and tempeh, are considered efficient strategies to reduce the demand for animal farming.

More recently, new food structures that resemble meat have been developed, the so-called meat analogues. Meat analogues are food structures produced from non-animal origin and that mimic the texture, taste and/or smell of meat. Meat analogues can be produced directly from microbial biomass or cell cultures, for example the commercial products developed using mycoprotein under the brand Quorn® or cultured meats developed by companies such as Mosa Meat®. Meat analogues can also be produced from a broad range of protein sources and hydrocolloids using well established technologies such as electrospinning, wet spinning, low and high moisture extrusion, freeze structuring and shear cell (Deckers et al., 2018). In order to create a meat-like texture, protein and fibre ingredients need to be processed under denaturing conditions in order to unfold and realign their polymer chains into a structure that resembles muscle tissue.

The development of new generation meat analogues requires novel ingredients that are sourced sustainably and which are minimally processed, rich in dietary fibres and low in nucleic acids. Typical plant-based ingredients are still limited by sustainability aspects related to water and land overexploitation. A problem with existing microbial derived ingredients, such as mycoprotein, is that they need to be processed significantly in order to reduce the nucleic acid content before they can be incorporated in food products.

Examples of microbial derived products include CN113317389A which describes a puffed food produced via extrusion, such product containing yeast protein and starch. The final product (75-85% protein and 15-25% starch) contains >60% protein and preferentially <1% RNA, water content of 5-25% with added calcium carbonate (0.05-0.1%) and white sugar (0.1-0.4%). Extrusion and expansion takes place at 120-140 deg C., screw frequency 60-80 Hz, and feed rate 6-8 Hz.

CN100469256C presents a method for transforming brewery yeast into peptides, involving the steps of extrusion of the biomass at 85-95° C. to reach a solid content of 45-55% at acidic (pH 4.5-4.8) conditions. This method also requires a series of thermochemical and enzymatic steps.

WO2013084064A2 describes a method for extruding legume flour such as soy and lupin, with 12-20% yeast autolysate and 1-30 ppm chromium and its use as medicament.

EP0212292A1 claims a foodstuff prepared with microbial protein isolate as a main ingredient (80-90%), obtained via extrusion at 160-180 rpm, 20-30 bar and 100-130° C.

The protein isolate can be obtained from obligate methylotrophs, after processing, separation and purification to remove fats and nucleic acids.

US2021392908A1, WO2021/195259, and WO2021/178254A1 describe a structured food product (meat analogue) produced via thermochemical processes (extrusion) from a microbial-derived product using a CO₂ fermenting organism, with 5-50% protein content in the microorganisms. The food product further contains 30-50% of the microbial protein product and a water content of 40-80%. However, multiple processing steps including RNA depletion and fat depletion are required.

CN109527198A presents a feed preparation in which a mixture of microbes is used to ferment a corn-based extrudate.

GB1144375A, JPH07106140B2, EP2047750A1, CN202244281U and GB2016996A describe methods and machines to produce yeast structures (compressed blocks, molded yeast, yeast granulate) and products containing yeast cells (e.g., noodles). Meanwhile, Caporgo et al., 2019, describes experiments to produce extrudates of algae and soy blends for meat analogues and pasta.

Although there are several protein rich microbial biomass and microbial derived products available, such products often require multiple processing, separation and purification steps, including defatting and RNA removal before they can be used in food preparations. It is therefore desired to develop simpler alternative methods that lead to microbial products that can be used as structuring ingredients.

### SUMMARY OF THE INVENTION

The present invention relates to a microbial cell extract, the incorporation of said extract in blends and processing said blends into a range of compositions, and use of said compositions. In one embodiment said composition has a meat like structure. In one embodiment said compositions may be used in food or any other suitable application.

Described herein is a method of preparing a microbial cell extract, said method comprising: providing a microbial biomass; subjecting said microbial biomass to disintegration; subjecting the resulting disrupted biomass to solid-liquid classification; optionally combining a portion of the heavy and light fractions obtained during classification. Said method may also optionally comprise a polishing step and/or concentration and/or drying step.

In an aspect of the invention, there is provided a method of preparing a microbial cell extract, the method comprising:
a) providing a microbial biomass in an aqueous alkaline suspension, at pH 7-11;
b) mechanically disintegrating the microbial biomass at a temperature below 35 deg C. using a non-denaturing process, such that the disintegrated biomass consists of a population of soluble compounds and suspended fragments characterized by a bimodal distribution and a particle size distribution (psd) at an average of around D50<4.5 μm;
c) subjecting the disintegrated biomass to a solid-liquid separation process, to separate the disintegrated biomass into an extract rich in small fragments and an extract rich in large fragments, wherein the extract rich in small fragments consists of a population of soluble compounds and suspended fragments characterized by a bimodal distribution and a psd of D50 around 0.5 um or lower; and the extract rich in large fragments consists of a population of soluble compounds and suspended fragments having a psd of D50>0.5 um.

In an another aspect of the invention, there is provided a method of preparing a microbial cell extract, the method comprising:
a) providing a microbial biomass in an aqueous alkaline suspension, at pH 7-11;
b) mechanically disintegrating the microbial biomass at a temperature below 35 deg C. using a non-denaturing process, such that the disintegrated biomass consists of a population of soluble compounds and suspended fragments characterized by a bimodal distribution and a particle size distribution (psd) at an average of around D50<4.5 μm;
c) subjecting the disintegrated biomass to a solid-liquid separation process, to separate the disintegrated biomass into an extract rich in small fragments and an extract rich in large fragments, wherein the extract rich in small fragments consists of a population of soluble compounds and suspended fragments characterized by a bimodal distribution and a psd of D50 around 0.5 um or lower; and the extract rich in large fragments consists of a population of soluble compounds and suspended fragments having a psd of D50>4 um.

The method may further comprise the step of:
d) recombining a portion of the extract rich in small fragments and a portion of the extract rich in large fragments to provide a microbial extract.

The ratio of the extracts to be recombined may be selected to provide desired properties, as will be described further herein.

Microbial biomass, which comprises microbial cells, has been used traditionally to produce a broad range of products of industrial interest, or has been used directly in a number of applications. Most industrial or commercial applications make use of a selected group of microbial biomass strains from the domains bacteria, yeast, fungi and algae. In overall terms, products obtained from microbial biomass are either intracellular or extracellular. Extracellular products are excreted by the cells into the bulk medium, usually an aqueous phase. Intracellular products, on the contrary, remain inside of the cells. In order to obtain intracellular products, additional processing is needed to release these products from the cells (by breaking the cell membrane or wall) and to further separate the compounds of interest from the remaining biomass and other impurities.

In the particular field of food applications, microbial biomasses have been used as a source of proteins (single cell protein—SCP), as nutritional supplements, or to produce various ingredients and additives.

Microbial biomasses are often used in the form of extracts, for which the microbial cells forming said biomass need to be disrupted/disintegrated. Extracts prepared from several different starting materials are known, such as fungal extract, algae extract and yeast extract. Of these extracts, the most commonly used is that derived from yeast, the so-called yeast extracts (hereinafter referred to as “YE”). YE are (and can be) applied in a broad range of products ranging from growth media for culturing cells for laboratories to nutritional supplements and flavor enhancers for the food industry. Production processes of YE are well-known. In general, yeast cells, mostly from the genus Saccharomyces, are disrupted by means of heat induced or chemically induced autolysis (or plasmolysis), followed by a step of incubation at high temperatures (>50° C.) in order to activate endogenous enzymes, which break down the large intracellular products such as proteins and nucleic acids into smaller components thereof such as peptides, amino acids and nucleotides. The digested slurry that is obtained is then further purified and supplemented—depending on the final application—to provide an extract that can be commercialized as YE.

The microbial biomass used in the present invention can be obtained from several microbial types, including microalgae, yeast, bacteria and fungi. Examples of genera from which the microbial biomass may be derived for the microbial extract to be produced are Saccharomyces and Pichia (yeast), Tetraselmis, Chlorella, Arthrospira (algae), Fusarium (fungi), Methylobacterium (bacteria) and Lactobacillus (bacteria). Preferably the microbial biomass is derived from yeast, more preferably from the genus Saccharomyces and/or Pichia. Yeasts which may be used in the present invention include Saccharomyces, such as S. cerevisiae, S. chevalieri, S. boulardii, S. bayanus, S. italicus, S. delbrueckii, S. rosei, S. micro-ellipsodes, S. carlsbergensis, S. bisporus, S. fermentati, S. pastorianis, S. rouxii, or S. uvarum; a yeast belonging to the genus Schizo-saccharomyces, such as S. japonicus, S. kambucha, S. octo-sporus, or S. pombe; a yeast belonging to the genus Hansenula, such as H. wingei, H. arni, H. henricii, H. americana, H. canadiensis, H. capsulata, or H. polymorpha; a yeast belonging to the genus Candida, such as C. albicans, C. utilis, C. boidinii, C. stellatoidea, C. famata, C. tropicalis, C. glabrata, or C. parapsilosis; a yeast belonging to the genus Pichia, such as P. pastoris, P. kluyveri, P. polymorpha, P. barkeri, P. cactophila, P. rhodanensis, P. cecembensis, P. cephalocereana, P. eremophilia, P. fermentans, or P. kudriavzevii; a yeast belonging to the genus Kluyveromyces, such as K. marxianus; and a yeast belonging to the genus Torulopsis, such as T. bovina, or T. glabrata.

In a preferred embodiment the microbial biomass is free from polluting material—for example, the biomass may be purified by centrifugation followed by washing and resuspension; several rounds of washing and resuspension may be used. In another embodiment said microbial biomass is prepared in an aqueous alkaline suspension (pH 7-11) at concentrations ranging from 5 to 15% DW. Optionally the pH is 8-10, or 8.5-9.5. Optionally the concentration is 6-14% DW, or 7-13%, 8-12%, 9-11% or 10% DW.

Generally, cell disintegration methods can be classified as being either non-mechanical or mechanical. Non-mechanical disintegration methods can be further sub classified into three categories: physical disintegration (e.g. by means of decompression, osmotic shock, thermolysis, ultrasounds, or freezing and thawing), chemical disintegration (e.g. by use of solvents, detergents, chaotropes, acids and bases, or chelates) and enzymatic disintegration (e.g. by autolysis, phage lysis, or lytic enzymes). The present invention is preferably related to mechanical disintegration methods. Examples of mechanical disintegration methods are ball mills, including bead mills, and homogenizers.

Ball mills (including bead mills) can be either vertical and horizontal and use a grinding medium which is present in the grinding chamber. A motor drives a rotor to rotate the cell suspension at a high speed. The cell suspension and the grinding material (e.g. beads) generate shearing force to break the cells. This results in the release of intracellular materials into the aqueous suspension and will also result in cell fragmentation (i.e., disintegration). With increasing rotor speed, the shear force increases and the cell breakage increases. With decreasing grinding material size, the cell breakage usually increases. Other parameters affect the performance of the disintegration process. The skilled person is capable of selecting the right parameters and variables in accordance with the present invention.

Homogenizers work under high-pressure and are in fact a positive-displacement pump that forces a cell suspension through a valve, before impacting the stream at high velocity on an impact ring. Often, several passes at high-pressure are required, which may lead to rising temperatures causing local denaturation of labile molecules.

Preferably the mechanical disintegration step is performed using bead milling or high pressure homogenization. Most preferably the mechanical disintegration step is performed using bead milling. Preferably, the disintegration step is carried out at a pH in the range of 7-11 (optionally 8-10, or 8.5-9.5), and at a temperature in the range of approximately 10-30° C., more preferably approximately 15-25° C., even more preferably 20-25° C., and most preferably <25° C. Carrying out the disintegration step within the said pH and temperature ranges has the technical effect of preventing the denaturation of proteins and other labile molecules (that is, it is a non-denaturing process) and consequently preventing the activation of lytic enzymes, proteases or other hydrolytic enzymes present in the microbial biomass. The skilled person is able to adjust the process parameters of the disintegration method (speeds, flows, filling ratios, bead sizes, pressures, etc) in order to keep the temperature preferably below <25° C. and to reach the desired psd target.

The disintegration step results in the production of a population of soluble compounds and suspended fragments characterized by a bimodal distribution and a particle size distribution (psd) at an average of around D50<4.5 μm. It is recognized that the skilled person would be able to adjust the parameters used during the disintegration step accordingly to achieve the desired targets of their application. During the course of the cell disintegration process, the psd varies, showing a decrease in the peak of intact cells, and a consequent increase in the peak of cell fragments. The disintegration process is run until a specific psd is obtained. For the example case of yeast, the specific surface area increases over time, while the D[3,2], D[4,3], D10, D50, D90 decrease over time. Other psd parameters may also be considered; for example, for yeast, one desired target psd may be D10<0.5 um, D50<4.5 um, D90<7.5 um and D[3,2]<2 um, D[4,3]<4.5 um.

In the art, particle size distribution is reported as a volume distribution. Taking D50 as an example it is known in the art that this can also be referred to as Dv50; the terms D50 and Dv50 are used interchangeably herein. The D50 or Dv50 is defined in the art as the maximum particle size, measured by diameter, below which 50% of the sample volume exists, also known as the median particle size (diameter) by volume. This concept is illustrated in FIG. 12. Numerous analytical techniques and approaches exist for particle size analysis.

A particle size analyser is an analytical instrument that measures, visualises, and reports a particle size distribution for a given particle or droplet population. Laser diffraction particle size analysers calculate particle size from the angle of light scattered by a stream of particles passing through a laser beam. This technique allows for continuous measurement of bulk material across a wide size range. The size limits and sensitivity of a laser diffraction particle analyser depend on the number and placement of detectors in the instrument. Dynamic light scattering particle analysers are mainly used for analysing particles in solution. Dynamic light scattering determines size from the fluctuations in scattered laser light intensity created by the particles' Brownian motion. Induced grating particle size analysers identify the size of small particles in solution by electrically aligning the particles and then measuring their diffusion.

In the context of the present invention the D50 was determined using a laser diffraction particle size analyser, the Malvern Mastersizer 2000, with a dispersant RI of 1.33 and a particle/material RI of 1.34, using a general-purpose analysis Model MS2000, and the Mie scattering model.

Disintegration methods are used to obtain a bimodal distribution and a particle size distribution (psd) at an average of around D50<4.5 μm as described above. Examples of cell disintegration methods according to the prior art are the following. U.S. Pat. No. 3,888,839A discloses a process for obtaining a protein isolate from yeast cells, wherein the yeast cells are ruptured by high-pressure homogenization (mechanical disintegration) and subsequent incubation. EP1199353A1 discloses a process for producing yeast extracts by treating yeast suspensions or yeast pastes and separating off the insoluble constituents, in which the yeast suspensions or yeast pastes are subjected to high-voltage electrical pulses (physical disintegration). EP2774993A1A discloses the use of a cell wall-decomposing enzyme (enzymatic disintegration) that does not contain protease and then heat-treating the product for 10 to 20 minutes at 70-80° C.

Microbial cells present in microbial biomass suspensions contain mostly proteins, carbohydrates, lipids and minerals. Proteins and other labile molecules experience unfolding, denaturation and degradation when exposed to high temperatures, long incubation times, extreme values of pH, solvents, salts and other harsh chemicals. When proteins and other functional molecules are denatured (tertiary and quaternary structure is lost), (part of) their functional activity is lost. Upon denaturation (unfolding), proteins lose their ability to interact with hydrophilic and hydrophobic surfaces, and also their ability to rearrange and form network-like structures upon heat-cooling treatments is affected. The present inventors have observed that the use of mechanical disintegration at the conditions described herein (for example, at the stated pH range) is sufficiently gentle to prevent unfolding, denaturation and/or degradation of proteins and other labile molecules, and therefore, necessary to preserve the functional properties, in particular gelation behavior, water holding capacity and oil holding capacity.

The aqueous suspension comprising microbial biomass may further comprise cytoplasmic material or other extracellular material produced during propagation or fermentation.

In an embodiment of the method according to the present invention, the microbial biomass comprises microbial cells selected from unicellular or colonial prokaryotes and eukaryotes and one or more combinations thereof. In a preferred embodiment, the microbial cells are selected from the group consisting of yeast, algae, bacteria, fungi, and one or more combinations thereof. In a specific embodiment, the microbial cells are yeast.

Bead sizes that may be considered are in the range of 0.1-5 mm, preferably in the range of 0.5-1 mm. Suitable bead materials include, but are not limited to, zirconium and glass. Bead fillings (the percentage of the bead mill chamber that is filled with beads) that may be considered suitable are in the range of 40-90%, preferably in the range of 65-80%, more preferably 75%, based on the total available volume of the bead mill chamber.

Rotational speeds that may be considered suitable are in the range of 1-20 m/s. Depending on the configuration and geometry of each bead mill, the skilled person can estimate the corresponding rotor speeds in rpm. Suitable rotational speeds in rpm are for example 500-5000 rpm, preferentially 1000-3000 rpm.

Concentration of microbial cells that may be considered suitable are in the range of 2-25% dry weight.

In one specific embodiment, microbial cells are disintegrated using a Dyno-mill Research Lab (CB Mills) bead mill. Cells can also be disrupted by shear forces, such as with the use of blending (such as with a high speed or Waring blender as examples), the French press, or even centrifugation in case of weak cell walls, to disintegrate cells.

In an embodiment, cell disintegration takes place without the addition of chemicals and/or solvents.

In a preferred embodiment the solid-liquid separation step is selected from a method known in the art including, but not limited to, centrifugation, decantation and filtration. More preferably, separation is performed by centrifugation, in a most preferred embodiment, separation is performed using centrifugation with a mild centrifugal field. An example of medium intensities is separation for <15 min at <4000 rcf in a bench top centrifuge. In an embodiment, said separation step is centrifuging at a centrifugal force equal to or smaller than 4000 relative centrifugal force (rfc). In an embodiment, said centrifuging takes place for a period of time equal to or shorter than 20 min. In a specific embodiment, said centrifuging takes place for a period of time equal to or shorter than 15 minutes. It will be understood that the skilled person is able to adjust the centrifugation parameters, depending on the type of equipment used, to reach the target characteristics (for example, psd) in both extracts.

The solid-liquid separation step results in the production of a light fraction (extract rich in small fragments) and a heavy fraction (extract rich in large fragments), as described in the patent application PCT/EP2021/075137 (WO2022/058287).

Preferably the light fraction is characterized by having a bimodal distribution with fragments>1 um and D50˜0.5 um. Furthermore, the volumetric ratio of solids to total suspension is at least 0.65 and preferentially >0.9.

Preferably the heavy fraction is characterized by having a D50>0.5 um, a protein content>30% DW and <55% DW, with at least 30% DW of total dietary fiber.

Alternatively, the heavy fraction is characterized by having a D50>4 um, a protein content>30% DW and <55% DW, with at least 30% DW of total dietary fiber

In a preferred embodiment a portion of the light fraction and a portion of the heavy fraction are combined to give a microbial cell extract with the desired properties. The skilled person would be able to adjust the amount of the heavy fraction and light fraction that are combined to provide a microbial cell extract with the desired properties.

In one embodiment the heavy and light fractions are recombined at a volumetric ratio of at least 0.65 heavy fraction to light fraction.

In one embodiment the heavy and light fractions are recombined at a volumetric ratio of at least 0.9 heavy fraction to light fraction.

In a preferred embodiment the recombined microbial cell extract has a protein content between 30-60%, more preferably between 35-50%, preferably 40-45%.

In another embodiment the recombined microbial cell extract has a total dietary fiber content of >20%, preferably >30%, more preferably 30-35%. Preferably the microbial cell extract has a protein content between 30-60% and a total dietary fiber of >20%. More preferably the microbial cell extract has a protein content between 35-50% and a total dietary fiber of >20%.

In one embodiment the recombined microbial cell extract has an RNA content<5%. In another embodiment the recombined microbial cell extract has a fat content<10%. In a preferred embodiment the recombined microbial cell extract has an RNA content<5% and a fat content<10%.

In one embodiment the recombined microbial cell extract has <4% RNA. In another embodiment the heavy fraction comprises ˜3% DW fat. In a preferred embodiment the heavy fraction comprises <4% RNA and ˜3% DW fat. This has the technical advantage of not requiring additional steps for fat depletion and/or RNA depletion.

In one embodiment the recombined microbial cell extract can absorb at least 2 times its own dry weight in water (in other words, it has a water holding capacity of at least twice its own dry weight). In another embodiment the recombined microbial cell extract can absorb at least 1.6 times its own dry weight in oil. In a preferred embodiment the recombined microbial cell extract can absorb at least 2 times its own dry weight in water and at least 1.6 times its own dry weight in oil.

The recombined microbial cell extract, and/or the separated heavy and/or light fractions either individually or together, may optionally be subjected to processing steps known in the art in order to improve the purity or properties of the extract. Example of such processes are filtration, adsorption, centrifugation, aqueous washing and alkaline washing. However, the microbial cell extract may be used directly without any additional processing steps.

In an embodiment, said processing step is membrane filtration, wherein the cut-off value of the membrane used is in the range of 1 kDa to 20000 kDa, preferably in the range 10 kDa to 1000 kDa. In an embodiment, the cut-off value of the membrane used is in the range of 0.1-2 μm.

The recombined microbial cell extract may optionally be dehydrated or dried. In a preferred embodiment the microbial cell extract is dried to a powder with a moisture content<8%. More preferably a drying method that yields a free-flowing powder is preferred. The method of the optional dehydration or drying step is selected from a method known in the art. Examples of drying technologies are spray drying, freeze drying, box dryer and ring dryer. The skilled person is able to select the right technology and processing conditions in order to obtain a free-flowing powder without compromising the technical functionality of the microbial cell extract.

The microbial extract of the present invention exhibits a unique viscoelastic behaviour under a heating cooling profile, as shown in FIG. 4. In one embodiment, during the heating phase, above ˜40° C., the viscosity and storage modulus (G′) increases until a plateau phase is reached at ˜80° C. Further, during the cooling period, G′ further increases, displaying a heat-set gelling behaviour, reaching a maximum G′max at room temperature (˜25° C.). It follows then that the elastic behaviour is the larger contributor to the overall rheological behaviour of the microbial extract of the present invention.

The microbial extract may be used in several compositions in order to prepare several structures. Preferably by structures it is meant herein that a molecular arrangement such that at macroscale a meat-like texture and sensory experience is achieved. It is therefore a primary aspect of the present invention to prepare structures that can be applied in food products. For example, the structure may reproduce a texture and/or organoleptic characteristic of natural meat. In some embodiments, the structure mimics the structure of ground or muscle meat. In certain embodiments, the structure includes one or more flavorings. In some embodiments, the structure is supplemented with one or more substances, such as vitamin, nutrient, or substance with a beneficial functional property. For example, the supplemental substance(s) may include one or more of amino acids, lipids, oils, fatty acids, vitamin B12 or other vitamins, biotin, antioxidants, minerals, surfactants, and emulsifiers.

In one embodiment the microbial cell extract may be subjected to thermochemical transformation to produce structures. Said thermochemical transformation may be conducted using extrusion, at high water content (high moisture extrusion), at low water content (low moisture extrusion). Alternatively, the microbial cell extract may be transformed using other methods known in the art including, but not limited to, spinning, electrospinning, power heater and 3d printing.

In a preferred embodiment thermochemical transformation processes of the microbial cell extract have a melting temperature in the range of 120-140° C., more preferably ˜126° C.

In a preferred embodiment, the microbial extract can be processed directly into structures using methods and technologies known in the art. An example of such technology is extrusion, more specifically low moisture extrusion and high moisture extrusion. High moisture extrusion is the preferred embodiment of the present application. In one embodiment the structures are meat-like structures (meat analogues) that can be sliced, cut, ground, shredded, grated, or otherwise processed, and that resemble animal-derived meat such as but not limited to beef, pork, chicken, fish or lamb, in applications that include, but are not limited to filet, burger, sausages, balls and steaks.

In a preferred embodiment the microbial extract can be used in compositions that can be processed into structures, where said structures display better attributes than the structures prepared using compositions without the microbial extract. Preferably, the microbial extract improves the properties of the structures prepared with said compositions. Moreover, in one embodiment of the present invention a composition that is not suitable to be processed into structures, becomes suitable to be processed into structures after the microbial extract is included in the composition. In the present invention the word composition is used to describe ingredients or components, or blends of ingredients or components. Non-limiting examples of such ingredients or components are protein and fibre rich fractions from soy, pea, fava bean, lentil, wheat, bran, rice, oat, barley and citrus. In a preferred embodiment said compositions can be formulated into food products. More preferably said compositions can be formulated into meat-like food products including but not limited to, chicken fillets, chicken burgers, hamburgers, bacon, steak, lamb fillets, pork fillets, sausages, meat balls, gammon steaks or mincemeat.

In one embodiment when the microbial cell extract is included in a composition with or without other protein rich and/or fibre rich ingredients such as soy, pea, fava bean, lentil, wheat, bran, rice, oat, barley and citrus, the composition including the microbial cell extract is lighter, less dense and better resembles an open fibrous meat like structure than compositions that do not include the microbial cell extract.

In one embodiment when the microbial cell extract is included in a composition with or without other protein rich and/or fibre rich ingredients such as soy, pea, fava bean, lentil, wheat, bran, rice, oat, barley and citrus, the composition including the microbial cell extract is less brittle, less dry and has superior tensile strength than compositions that do not include the microbial cell extract.

In one embodiment, the microbial cell extract is combined with at least one leavening agent. A leavening agent is also known in the art as a raising agent and can be defined as an agent that creates a foaming action or gas bubbles that lighten a mixture. Examples of leavening agents that may be combined with the microbial cell extract include but are not limited to sodium bicarbonate, ammonium bicarbonate, potassium bicarbonate, potassium bitartrate and sodium aluminium phosphate. Preferably said leaving agent is sodium bicarbonate. More preferably the leavening agent is sodium bicarbonate combined with the microbial cell extract at 0.1% wt/wt. The skilled person would be able to select any leavening agent known in the art and adapt the amount to be combined with the microbial cell extract based on the application and user's needs.

In another embodiment, the microbial cell extract is combined with at least one sulphur containing compound. A non-limiting example of a sulphur containing compound that may be combined with the microbial cell extract is sulphur containing amino acid. Preferably, the microbial cell extract is combined with sulphur containing amino acids at 0.1% wt/wt.

In another embodiment, the microbial cell extract is combined with at least one additive and/or ingredient such as EDTA, citric acid or acetic acid. An additive is any substance that is added to food during processing or production to improve its quality, appearance, flavour, texture, or shelf life. Additives can be natural or synthetic. The skilled person would be able to select any additive and/or ingredient known in the art and adapt the quantity to be combined with the microbial cell extract depending on the application and user's needs.

In an embodiment, the microbial cell extract is included in a composition including one or more of a leavening agent, a sulphur containing compound, an additive and/or ingredient.

In a preferred embodiment the microbial cell extract is included in a composition to account for 20-40% dry weight wherein the pH is preferably alkaline, more preferably at a pH of ˜8.5.

In a preferred embodiment the microbial cell extract provides an umami taste when included in structures produced from compositions including the microbial cell extract.

In a preferred embodiment structures produced from compositions including the microbial cell extract in addition to other protein rich and/or fibre rich ingredients can be hydrated and/or blanched at a comparable or faster speed than compositions that do not comprise the microbial cell extract.

In a preferred embodiment structures containing the microbial cell extract resemble meat-like structures when hydrated, blanched, baked or fried. Said meat-like structures may then be sliced, cut, ground, shredded, grated, or otherwise processed, and resemble animal-derived meat such as but not limited to beef, pork, chicken, fish or lamb, in applications that include, but are not limited to filet, burger, sausages, balls and steaks.

The structures produced from compositions containing the microbial extract display at least one unexpected attribute compared to structures lacking the microbial extract. Examples of said unexpected attributes are as follows:
Structures are less dense
Structures are less brittle and less dry
The tensile strength of the structures is superior
The hydration time of the structures is significantly shorter
The blanching times of the structures is shorter
The structures present a more pleasant appearance
Good structures are obtained at specific processing conditions (e.g., Tm) and narrow inclusion levels in the compositions (e.g., 20-40%)
The properties of the structures are better when the pH of the composition is adjusted to ˜8.5

In one embodiment the present invention is characterized by a unique simple process to obtain a microbial extract. In another embodiment the present invention is characterized by the unexpected excellent performance of said microbial extract in producing structures. In a further embodiment the present invention is characterised by the applications of such structures in for example food products.

A further aspect of the invention provides a method for preparing a substitute food composition, the method comprising:
a) providing a microbial biomass in an aqueous alkaline suspension, at pH 7-11;
b) mechanically disintegrating the microbial biomass at a temperature below 35 deg C. using a non-denaturing process, such that the disintegrated biomass consists of a population of soluble compounds and suspended fragments characterized by a bimodal distribution and a particle size distribution (psd) at an average of around D50<4.5 μm;
c) subjecting the disintegrated biomass to a solid-liquid separation process, to separate the disintegrated biomass into an extract rich in small fragments and an extract rich in large fragments, wherein the extract rich in small fragments consists of a population of soluble compounds and suspended fragments characterized by a bimodal distribution and a psd of D50 around 0.5 um or lower; and the extract rich in large fragments consists of a population of soluble compounds and suspended fragments having a psd of D50>0.5 um.
d) recombining a portion of the extract rich in small fragments and a portion of the extract rich in large fragments to provide a recombined microbial extract; and
e) extruding the recombined microbial extract to provide a substitute food composition.

Also provided by the invention is a recombined microbial extract, the extract comprising a protein content between 30-60%, more preferably between 35-50%, preferably 40-45%. Preferably the recombined microbial extract has a total dietary fiber content of >20%, preferably >30%, more preferably 30-35%.

Preferably the recombined microbial extract has an RNA content<5%. In another embodiment the recombined microbial cell extract has a fat content<10%. In a preferred embodiment the recombined microbial cell extract has an RNA content<5% and a fat content<10%.

In one embodiment the recombined microbial cell extract can absorb at least 2 times its own dry weight in water (in other words, it has a water holding capacity of at least twice its own dry weight). In another embodiment the recombined microbial cell extract can absorb at least 1.6 times its own dry weight in oil. In a preferred embodiment the recombined microbial cell extract can absorb at least 2 times its own dry weight in water and at least 1.6 times its own dry weight in oil.

A preferred recombined microbial cell extract thus comprises:
a protein content between 30-60%,
a total dietary fiber content of >20%
an RNA content<5%
a fat content<10%
has a water holding capacity of at least twice its own dry weight; and
has an oil holding capacity of at least 1.6 times its own dry weight.

The invention further provides a substitute food product comprising the microbial cell extract described herein. The substitute food product may be a substitute meat product. The substitute food product preferably has a molecular arrangement such that at macroscale a meat-like texture and sensory experience is achieved. For example, the substitute food product may reproduce a texture and/or organoleptic characteristic of natural meat. In some embodiments, the substitute food product mimics the structure of ground or muscle meat. In certain embodiments, the substitute food product includes one or more flavorings. In some embodiments, the substitute food product is supplemented with one or more substances, such as vitamin, nutrient, or substance with a beneficial functional property. For example, the supplemental substance(s) may include one or more of amino acids, lipids, oils, fatty acids, vitamin B12 or other vitamins, biotin, antioxidants, minerals, surfactants, and emulsifiers.

Also provided is a food ingredient comprising the microbial cell extract described herein. The food ingredient may be in the form of a powder, flour, paste, gel.

Although primarily described herein with reference to preparations obtained from yeast cells, the invention is not limited to the same. Various other microorganisms can be used.

In embodiments, the microbe may be selected from fungi, including yeast (preferably Saccharomyces sp, more preferably brewer's or baker's yeast, or Pichia sp); plants, in particular microalgae (including Tetraselmis sp or Chlorella sp, for example C. vulgaris); and cyanobacteria (including Arthrospira sp, preferably A. platensis). The microbe may also be selected from bacteria, for example lactic acid bacteria or Methylobacterium spp.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage.

The scope of the present invention is defined by the appended claims. One or more of the objects of the invention are achieved by the appended claims

### List of Definitions

The following definitions are used in the present description and claims to define the stated subject matter. Other terms not cited below are meant to have the generally accepted meaning in the field.

“Drying” as used in the present description means reducing the moisture content. The term drying includes partial drying wherein moisture may remain after drying in a reduced amount, which can also be seen as concentrating.

“Dry weight (DW)” and “dry cell weight” as used in the present description mean weight determined in the relative absence of water. For example, reference to microbial biomass as comprising a specified percentage of a particular component by dry weight means that the percentage is calculated based on the weight of the biomass after substantially all water has been removed.

“Disruption” as used in the present description in the context of microbial cells is also referred to as “lysing” and means opening the cells to release cytoplasmic compounds (also referred to as the “lysate”).

“Disintegration” as used in the present description means, in the context of disintegration of microbial cells, the fragmentation of the cells. This implies that the average size of the resulting cell fragments must be smaller than the average cell size of the initial microbial cells. Disintegration can be seen as a specific type of disrupting in which not only the cells are opened, but in which the cells are also fragmented.

“Cytoplasmic material” or “Cytoplasmic compounds” as used in the present invention means all material that is usually contained within a cell, enclosed by the cell membrane, except for the cell nucleus (if present). When a cell is disintegrated or disrupted, the cytoplasmic material is released from the cell.

“Microbial cells” as used in the present description means: microbes. This can be eukaryotic and prokaryotic unicellular organisms and colonies of them. A prokaryote is a cellular organism that lacks a membrane-enclosed nucleus. In the three-domain system, based upon molecular analysis, prokaryotes are divided into two domains: Bacteria (formerly Eubacteria) and Archaea (formerly Archaebacteria). Organisms with nuclei are placed in a third domain, Eukaryota. Microbial cells according to the present invention also encompass eukaryotes including algae and fungi such as yeast.

“Microorganism” and “microbe” as used in the present description mean any microscopic colonial or unicellular organism.

“Microbial cell product” as used in the present description means: a product derived from microbial cells that is obtained by processing microbial cells in a certain manner.

“Extract enriched in small cell fragments (EESF)” as used in the present invention means a microbial cell product that is obtained by separation of the aqueous suspension comprising disintegrated microbial cells. During this separation an extract is separated out of the aqueous suspension comprising disintegrated microbial cells leaving behind an aqueous suspension comprising disintegrated microbial cells that is partly depleted from small cell fragments. In other words, the separation treatment produces an extract enriched in small cell fragments (as main product) and an aqueous suspension depleted in small cell fragments, the latter can and will also be referred to as an extract enriched in large cell fragments. “Small cell fragments” as used in the present description means cell fragments obtained from disintegration of microbial cells having a size of equal to or less than d50≤500 nanometers (nm).

“Extract enriched in large cell fragments (EELF)” or “aqueous suspension depleted in small cell fragments” as used in the present invention means a microbial cell product that is obtained by separation of the aqueous suspension comprising disintegrated microbial cells. During this separation an extract is separated out of the aqueous suspension comprising disintegrated microbial cells leaving behind an aqueous suspension comprising disintegrated microbial cells that is partly depleted from small cell fragments.

In other words, the separation treatment produces an extract enriched in small cell fragments (as main product) and an aqueous suspension depleted in small cell fragments (as by product), the latter can and will also be referred to as an extract enriched in large cell fragments. “Large cell fragments” as used in the present description means cell fragments obtained from disintegration of microbial cells having a size more than d50≥500 nanometer (nm).

“Enriched” or “enrichment” as used in the present description means selective movement of particles to one of the two phases of separation i.e. the EESF or the EELF; this concept is illustrated in FIG. 13. When using high centrifugal forces, all particles/insoluble material are transferred to the heavy phase (FIG. 13b); if a centrifugal force is used that is too low the separation between the light and the heavy phase is poor and fragments are not clearly separated (FIG. 13d). However, using a mild or medium centrifugal force results in the small fragments being preferentially concentrated in the light phase (extract enriched in small fragments), while large fragments are preferentially concentrated in the heavy phase (extract enriched in large fragments), shown in FIG. 13c. The concept of enrichment is further described in example 12. Note that this is not the same as simply separating soluble and insoluble fractions, since insoluble material remains in both the EESF and EELF.

“Microbial biomass” and “biomass” as used in the present description mean a material produced by growth and/or propagation of microbial cells, or produced as byproduct of fermentation processes. Biomass may contain cells and/or intracellular contents as well as extracellular material. Extracellular material includes, but is not limited to, compounds secreted by a cell.

“Bead milling” as used in the present description means agitation of microbial cells in suspension with small abrasive particles (beads). Cells break because of shear forces, grinding between beads, and collisions with/between beads. Shear forces produced by the beads disrupt the cells and cause disintegration with concomitant release of cellular compounds.

“Centrifugation” as used in the present description means the application of centrifugal force to separate particles from a solution according to their size, shape, density, viscosity of the medium and rotor speed, among other parameters. The rate of centrifugation is specified by the angular velocity usually expressed as revolutions per minute (RPM), or acceleration expressed as g. The conversion factor between RPM and g depends on the radius of the centrifuge rotor. The general formula for calculating the revolutions per minute (RPM) of a centrifuge is

$$RPM = \sqrt{\frac{g}{r}}$$

where g represents the respective force of the centrifuge and r the radius from the center of the rotor to a point in the sample. However, depending on the centrifuge model used, the respective angle of the rotor and the radius may vary, thus the formula gets modified. The most common formula used for calculating Relative Centrifugal Force is:

$$RCF ( * g ) = 1.118 * r * {( \frac{RPM}{1000} )}^{2}$$

wherein r is the radius in mm.

“Water holding capacity (WHC)” as used in the present description refers to the amount of water a sample can hold per unit of weight.

“Oil holding capacity (OHC)” as used in the present description refers to the amount of oil a sample can hold per unit of weight.

“Viscoelastic” as used in the present description refers to a substance exhibiting both elastic and viscous behaviour when deformed.

### BRIEF DESCRIPTION OF DRAWINGS

FIG. 1—Particle size distribution and particle sizes of an aqueous suspension comprising microbial cell biomass after bead milling.

FIG. 2—Particle size distribution and particle sizes after classification of the disintegrated aqueous suspension comprising microbial cell biomass by centrifugation (green represents the heavy fraction and red represents the light fraction).

FIG. 3—FIG. 3a shows the composition of the microbial cell extract, the composition was analyzed 5 separate times and the mean and standard deviation was calculated, here Ins HMW represents insoluble high molecular weight fiber, Sol HMW represents soluble high molecular weight fiber, LMW represents low molecular weight fiber and DF represents dietary fiber. FIG. 3b shows the water and oil holding capacities of said microbial cell extract.

FIG. 4—Viscoelastic behaviour of a 20% DW microbial cell extract suspension analysed under a heating-cooling profile.

FIG. 5—Determination of melting temperature during extrusion

FIG. 6—Determination of preferable inclusion levels of microbial cell extract in a composition with pea protein isolate (PPI) and gluten

FIG. 7—Determination of preferable inclusion levels of microbial cell extract in different compositions

FIG. 8—Hydration times, blanching times and textures for structures prepared with several compositions

FIG. 9—Hydration times, blanching times and textures for structures prepared with several compositions including pea protein isolate (PPI) and gluten as reference protein rich ingredients.

FIG. 10—Hydration times, blanching times and textures of structures prepared with compositions including several reference protein rich and fiber rich ingredients.

FIG. 11—Appearance of structures prepared with microbial cell extract. FIG. 11A shows the appearance of HM extrudates containing SPI and microbial cell extract. FIG. 11B shows the appearance of LM extrudates containing SPC and microbial cell extract.

FIG. 12—Illustration of D10, D50, D90 for a bimodal distribution based on volume.

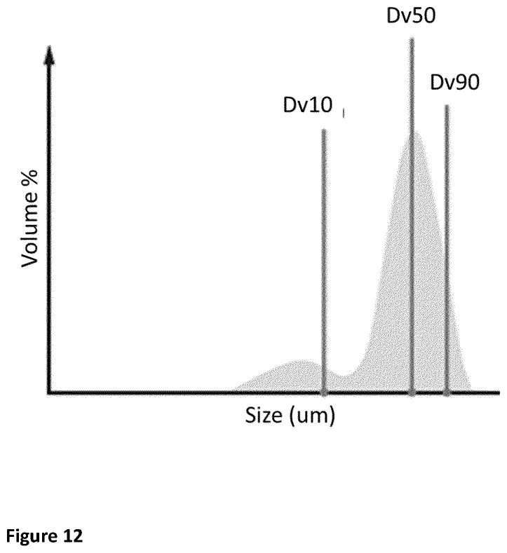

FIG. 13—Enrichment of particles from a disrupted suspension with a population of fragments (FIG. 13a) and separation into light and heavy phase under high centrifugal force (FIG. 13b), medium centrifugal force (FIG. 13c) and low centrifugal force (FIG. 13d). Phase split is indicated with a horizontal line.

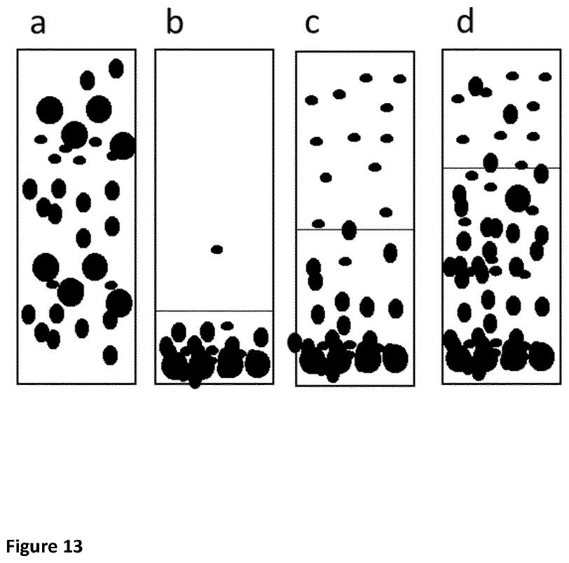

FIG. 14—Psd of yeast biomass (circles), disrupted biomass (squares), the fraction enriched in small fragments (triangles) and the fraction enriched in large fragments (inverted triangles) according to the invention.

FIG. 15—Psd of the light phase after cell disintegration and centrifugal separation at several intensities (g forces): high intensity of 20000×g for 15 minutes (triangles), medium intensity of 4000×g for 15 minutes (squares) and low intensity of 1000×g for 5 minutes (circles). The dashed square shows the desired range for particle enrichment.

FIG. 16—Gelation hardness for different recombination ratios of the EESF and the EELF according to the present invention.

FIG. 17—Psd of the disintegrated biomass, EESF and EELF derived from Methylobacterium spp. according to the present invention.

FIG. 18—Maximum extension resistance for extrudates after hydration and baking for blends prepared comprising commercial ingredients (fava bean protein isolate (FBI), fava bean protein concentrate (FBC), gluten, pea protein concentrate (PPC) and pea protein isolate (PPI)) and the microbial cell extract of the present invention.

### DETAILED DESCRIPTION OF EXAMPLES

### Examples

The invention is described herein by the following non limiting examples.

### Example 1

A microbial cell extract is produced according to the method described in PCT/EP2021/075137 (WO2022/058287). In summary, said microbial cell extract is produced by i) providing an aqueous suspension comprising microbial cells; ii) subjecting said suspension to mechanical cell disintegration, to obtain an aqueous suspension comprising disintegrated microbial cells; and iii) separating the suspension to provide an extract enriched in small cell fragments (“light phase”), and an extract enriched in large cell fragments (“heavy phase”). It is noted that optionally at least a portion of each extract may be recombined, to provide a recombined microbial cell product.

In this example the aqueous suspension comprising microbial cells comprises yeast biomass at ˜100 g/L adjusted to ˜pH 9 with NaOH and subjected to bead milling using 0.5-0.65 mm Zirconium beads, with a 75% filling rate, ˜12 m/s rotor speed, and batch recirculation mode, at a temperature of ˜23° C.

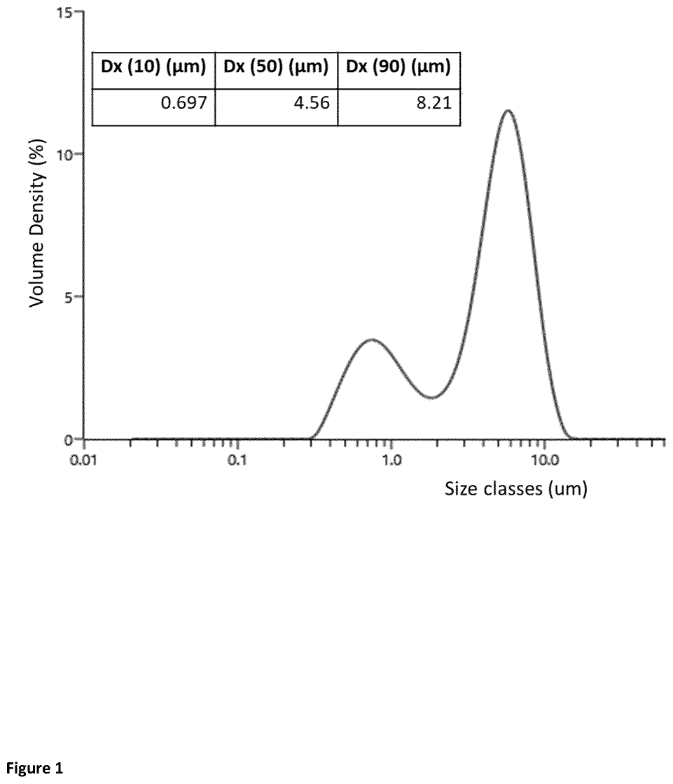

The resulting disintegrated biomass has a particle size distribution and particle sizes shown in FIG. 1. The disintegration process is run in such a way that a bimodal particle size distribution (psd) is obtained, displaying a peak of intact cells, and a peak of cell fragments. For the case of yeast, a peak of intact cells ˜6 μm and a peak of cell fragments ˜0.8 um is obtained.

During the course of the cell disintegration process, the psd varies, showing a decrease in the peak of intact cells, and a consequential increase in the peak of cell fragments. The disintegration process is run until a specific psd is obtained.

After the disintegration step, the resulting microbial suspension is subjected to centrifugation using a batch centrifuge at 4000×g for 15 minutes at 15° C. This results in the formation of a light phase and a heavy phase of the microbial suspension (also referred to as an extract enriched in small cell fragments, and an extract enriched in large cell fragments, respectively).

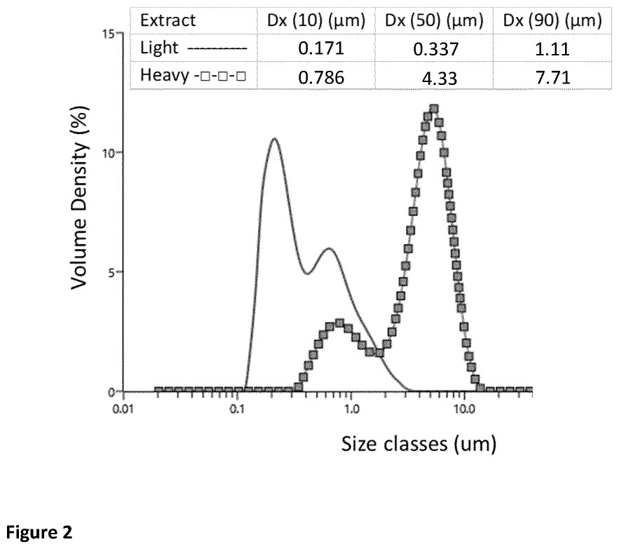

The particle size distribution and particle sizes of the light and heavy fractions resulting from separation by centrifugation are shown in FIG. 2 where the red line represents the light fraction and the green line represents the heavy fraction. The light fraction has its largest peak for cell fragments at ˜0.4 μm and another slightly smaller peak of cell fragments ˜0.8 um. The heavy fraction has its largest peak for intact cells ˜8 μm and a much smaller peak of cell fragments ˜0.8 um is also obtained. The phases have particle size distributions of:

| Extract | Dx (10) (μm) | Dx (50) (μm) | Dx (90) (μm) |
|---|---|---|---|
| Light | 0.171 | 0.337 | 1.11 |
| Heavy | 0.786 | 4.33 | 7.71 |

After separation by centrifugation the volumetric ratio of the heavy phase is as follows:
Volume feed (VF)=10 ml;
Volume heavy phase (VH)=8.0 ml;
Volume light phase (VL)=2.0 ml Volumetric ratio=8/10=0.8.
VH/VF=Volumetric ratio of heavy phase: 8/10=0.8
Volumetric ratio of heavy phase=0.8

### Example 2

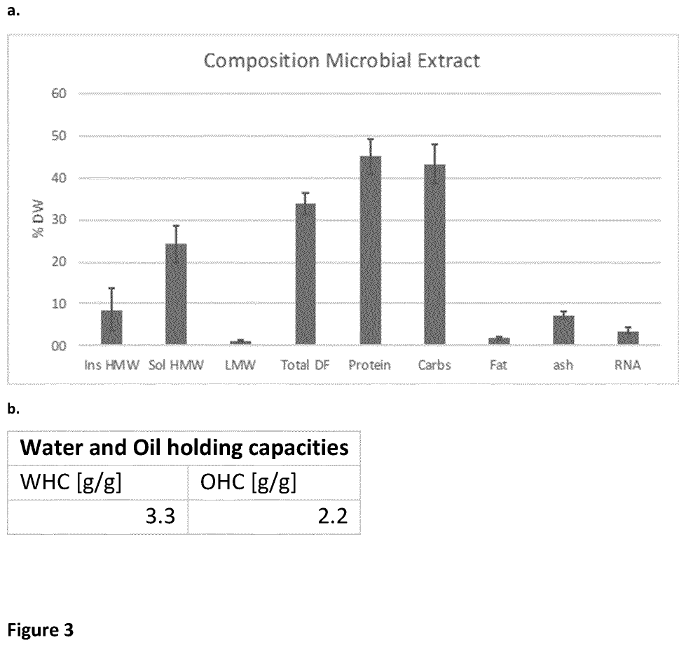

The heavy phase of a microbial cell extract produced according to the method described in Example 1 had its composition analysed, 5 measurements were taken and the mean and standard deviation were calculated. The composition of said microbial cell extract is shown in FIG. 3a. Here it can be seen that protein accounts for the largest proportion of the microbial cell extract, accounting for ˜45% DW whilst LMW, fat and RNA only accounts for very small components<10%.

The functional properties, water holding capacity and oil holding capacity were also measured for the microbial cell extract produced according to the method described in Example 1. The water and oil holding capacities are shown in FIG. 3b, the water holding capacity was highest at 3.3 g/g whilst the oil holding capacity was 2.2 g/g.

### Example 3

The rheological behaviour of the heavy phase microbial cell extract produced according the method set out in Example 1 was also measured, specifically the viscoelastic behaviour of a 20% DW microbial cell extract suspension was analysed under a heating-cooling profile.

Viscoelasticity is the property of materials that exhibit both viscous and elastic characteristics when undergoing deformation. Viscous materials, like water, resist shear flow and strain linearly with time when a stress is applied. Elastic materials strain when stretched and immediately return to their original state once the stress is removed. Viscoelastic materials have elements of both of these properties and, as such, exhibit time-dependent strain.

The storage modulus relates to the material's ability to store energy elastically. The loss modulus of a material is the ratio of the viscous (out of phase) component to the stress, and is related to the material's ability to dissipate stress through heat. If the storage modulus is higher than the loss modulus the material can be regarded as mainly elastic.

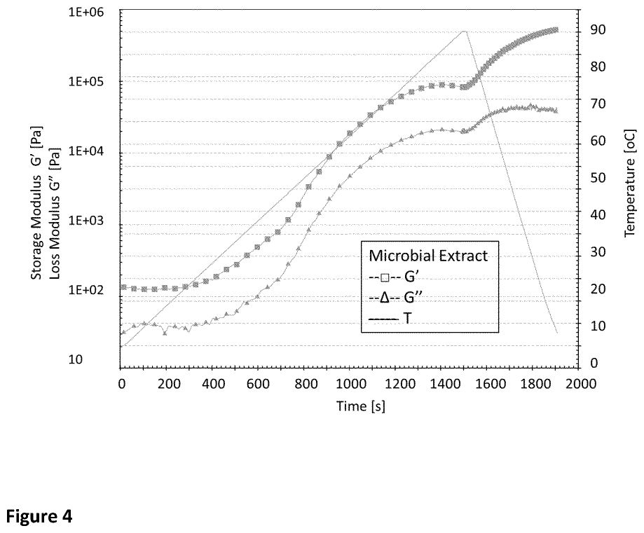

In FIG. 4 it can be seen that the storage modulus is higher than the loss modulus and therefore the microbial cell extract is mainly elastic.

### Example 4

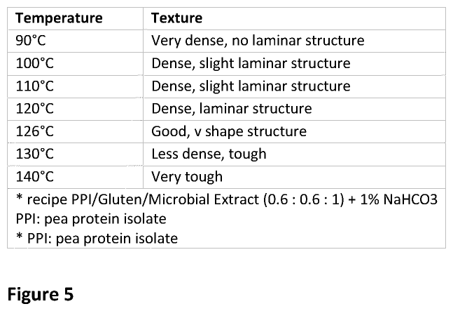

The heavy phase microbial cell extract produced according the method set out in Example 1 was subjected to temperatures between 90-140° C. to determine its melting temperature during extrusion. Specifically a composition comprising pea protein isolate, gluten and heavy phase microbial cell extract at a ratio of 0.6:0.6:1+1% NaHCO₃ was used. Here the temperature providing the optimal texture with a V shape structure was 126° C., this is shown in FIG. 5.

### Example 5

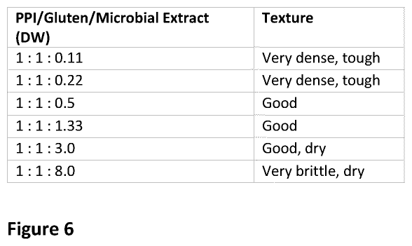

The preferable inclusion levels of the heavy phase microbial cell extract produced according the method set out in Example 1 was tested at a range of ratios set out in FIG. 6. Here it can be seen that a ratio of 1:1:0.5 and 1:1:1.33 (DW) pea protein isolate: gluten: microbial cell extract provided preferable textures.

### Example 6

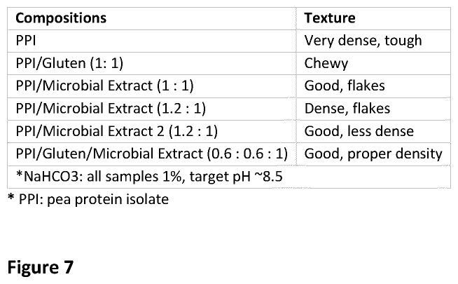

The effect of the inclusion of different levels of the heavy phase microbial cell extract produced according the method set out in Example 1 was analysed in numerous different compositions, set out in FIG. 7. Here is can be seen that compositions of pea protein isolate and microbial cell extract at 1:1, pea protein isolate and microbial cell extract 2 at 1.2:1 and pea protein isolate, gluten and microbial cell extract at 0.6:0.6:1 provided the most preferable compositions.

### Example 7

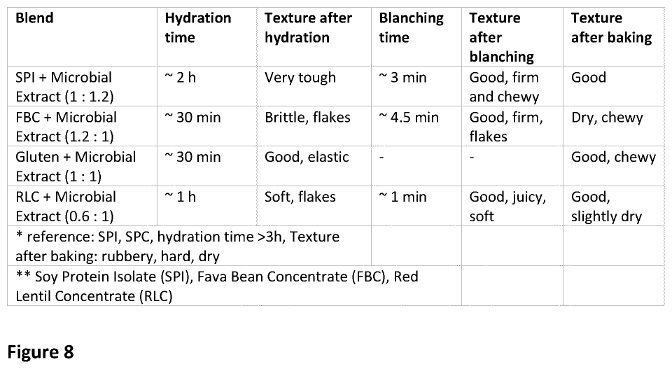

The hydration times, blanching time and textures for several compositions containing the heavy phase microbial cell extract produced according the method set out in Example 1 was analysed, as shown in FIG. 8. Here it can be seen that a composition of 1:1 gluten and microbial cell extract hydrated for 30 minutes provided a product with a good texture. A composition of red lentil concentrate (RLC) and microbial cell extract at 0.6:1 subjected to ˜1 hour hydration and ˜1 minute blanching also gave rise to a product with a good texture, although with a slightly dry texture after baking. A composition of soy protein isolate (SPI) and microbial cell extract at 1:1.2 subjected to ˜2 hours hydration and ˜3 minutes blanching gave rise to a product with a good texture, particularly after baking.

### Example 8

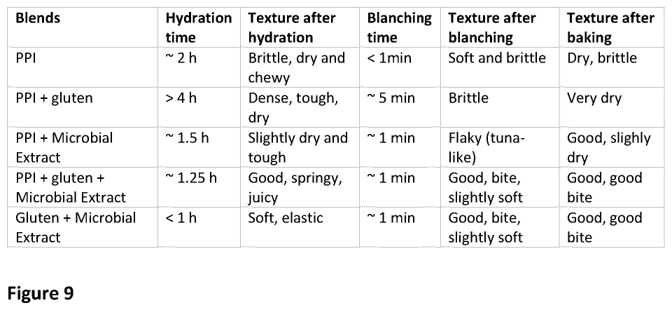

The hydration times, blanching time and textures for several compositions containing the heavy phase microbial cell extract produced according the method set out in Example 1 and pea protein isolate (PPI) and gluten as reference protein rich ingredients was analysed as shown in FIG. 9. Here it can be seen that the best textures were achieved with a composition of PPI, gluten and microbial cell extract hydrated for 1.25 hours and blanched for ˜1 minute, and with a composition of gluten and microbial cell extract hydrated for ˜1 hour and blanched for ˜1 minute.

### Example 9

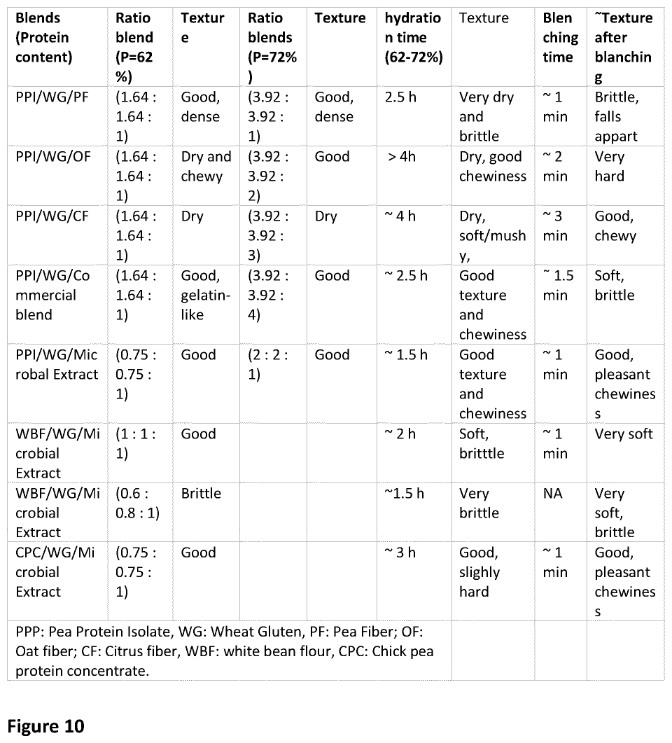

The hydration times, blanching time and textures for several compositions containing the heavy phase microbial cell extract produced according the method set out in Example 1 and several protein rich and fibre rich ingredients was analysed as shown in FIG. 10. Here it can be seen that the best textures overall were observed for a blend of PPI, WG (wheat gluten) and microbial cell extract at a ratio blend p=62% 0:75:0.75:1 and a ratio blend p=72% 2:2:1 hydrated for ˜1.5 hours and blanched for ˜1 minute as well as a blend of CPC (Chick pea protein concentrate), WG, and microbial cell extract at a ratio blend p=62% 0.75:0.75:1 hydrated for ˜3 hours and blanched for ˜1 minute.

### Example 10

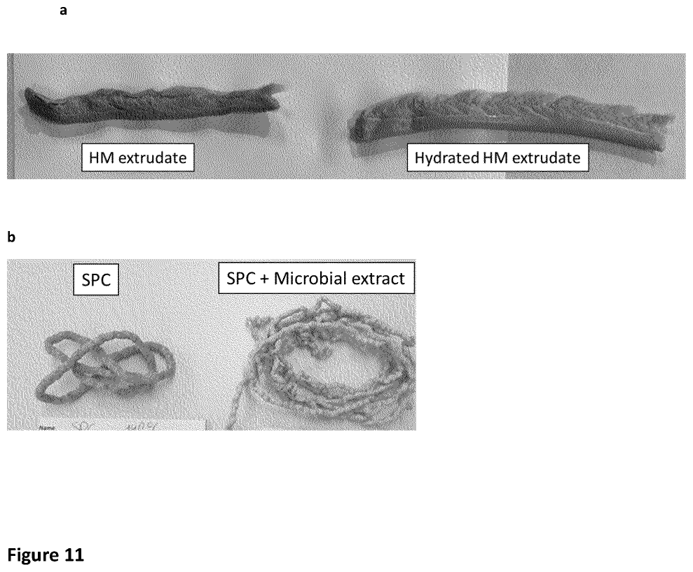

The appearance of structures prepared with the heavy phase microbial cell extract produced according the method set out in Example 1 is shown in FIG. 11. FIG. 11A shows the appearance of high moisture (HM) extrudates containing SPI and microbial cell extract. Here it can be seen that after 1 hour of hydration the density of the structure decreases, the colour become lighter, with the structure also being less brittle with the fibrous structure (V shape) also being more clearly visible.

FIG. 11B shows the appearance of low moisture (LM) extrudates containing SPC (soy protein concentrate) and microbial cell extract. Here it can be seen that extrudates containing microbial cell extract are less uniform but have superior tensile strength. Notably, brittleness is lower and hydration time is significantly faster. For microbial cell extract containing extrudate, extrusion takes place at 153° C. and 8 bar in comparison to the SPC extrudate that requires 120° C. and 40 bar.

### Example 11

Here examples of preparing food products using the structures obtained with mixtures of the light and heavy phase microbial cell extract of the present invention is described.

### 1. A Chicken Fillet Analogue

A chicken fillet analogue was prepared with the following ingredients
Low moisture extrudate (LME) and high moisture extrudate (HME) based on soy protein isolate and heavy microbial cell extract, as described herein
5 g of light phase microbial cell extract as described in patent application PCT/EP2021/075137 (WO2022/058287) wherein the light phase is enriched in cell fragments obtained from disintegration of microbial cells having a size of equal to or less than d505500 nanometers (nm); said light phase is then subjected to an additional polishing step (binding agent)
Methyl cellulose (MC) pre-hydrated
Cysteine (flavor enhancer)
4% bamboo Fibre
Sunflower oil

### Preparation Method:

Dry ingredients are mixed and blended into a dough made with the LME and HME extrudates in the pre-hydrated MC
The resulting dough is left resting at room temperature
The dough is baked or fried until slight browning is noticed on the surface and the internal structured is properly cooked.
The resulting structure has the texture and taste that resembles a chicken fillet from animal origin.

### 2. A Beef Burger Analogue

A beef burger analogue was prepared with the following ingredients
Low moisture extrudate (LME) and high moisture extrudate (HME) based on soy protein isolate and heavy phase microbial extract in a texturized vegetable protein (TVP) based on pea.
Methyl cellulose (MC) pre-hydrated
2% of light phase microbial extract prepared as above
Bamboo fibre
Beetroot red, caramel and salt as flavour and colour enhancers

### Preparation Method:

Dry ingredients are mixed and blended into a dough made with the LME and HME extrudates in the pre-hydrated MC and pre-hydrated TVP.
The resulting dough is left resting at room temperature
The dough is baked or fried until browning is noticed on the surface and the internal structure is properly cooked.
The resulting structure has the texture and taste that resembles a beef burger from animal origin.

### Example 12

Microbial cell extracts were prepared according to Example 1, the heavy phase was then subjected to drying. The resulting powder was then blended with commercial pea protein isolate (PPI), pea protein concentrate (PPC), pea fibre (PF). In some blends cysteine and NaHCO₃ were also included, as shown below in table 1.

The resulting blends were then subjected to thermo-physical modification by means of high moisture extrusion in a tween screw extruder at 130° C., the resulting textures water content was then analysed following the oven method, wherein the samples were kept at 100° C. in an oven until they are at a constant weight. The results are shown below in table 1.

TABLE 1
Moisture content of extruded blends (wt %)
with the composition defined below

| Composition of blend (wt %) | | | | | | |
|---|---|---|---|---|---|---|
| | | | Microbial | | | |
| | | | cell extract | | Moisture content | |
| | | | heavy | Cyst + | (wt %) | |
| PPI | PPC | PF | phase | NaHCO₃ | Extruded | Hydrated |
| 49 | 40 | 0 | 10 | 1 | 42.3% | 65.1% |
| 50 | 40 | 0 | 10 | 0 | 39.9% | 54.0% |
| 49 | 40 | 10 | 0 | 1 | 42.2% | 54.3% |
| 50 | 40 | 10 | 0 | 0 | 41.1% | 53.8% |

It is clear from table 1 that directly after extrusion the moisture content of the extrudates is comparable. However, after hydration, the moisture content of the extrudates containing the microbial cell extract with cysteine and NaHCO₃ was superior. This indicates that there is unique functionality of the microbial cell extract, and unexpected synergy with cysteine and NaHCO₃. A testing panel of 5 members was also consulted. This panel reported that both the extrudates comprising microbial cell extract, and microbial cell extract with cysteine and NaHCO₃ had a better mouthfeel, taste and texture compared to the reference extrudate containing PPI, PC and PF.

### Example 13

Qualitative observations were recorded from a testing panel of 5 individuals regarding the textures produced from high moisture extrusion in a tween screw extruder (temperature ˜126° C.) using blends of ingredients including the microbial cell extract heavy phase as described in example 1. For each blend tested a comparison was made to the same ingredients, however, without inclusion of the microbial cell extract. The feedback provided by the panel is summarised below.
a. Blend of the microbial cell extract combined with fava bean protein isolate/concentrate
   Less chewy (regarded as an improvement).
   More open texture.
   More elastic.
   Less earthy tones.
   Better/faster hydration.
   More robust texture (more resistant to falling apart).
b. Blend of the microbial cell extract combined with pea protein isolate/concentrate
   Presence of the microbial cell extract masked some of the off-taste of pea-based formulations and improved the colour to be more visually pleasing.
   The microbial cell extract improved the texture of the formulation, making it possible to develop gluten-free tuna-like textures.
   The microbial cell extract in specific combinations with pea and gluten provides chicken nugget like structures.
c. Blend of high and low moisture extrudates
   The microbial cell extracts subjected to high and low moisture extrusion can be recombined in specific ratios to yield unique textures that can be used for the development of different types of meat analogues.
d. Extrusion trials with several fibres
   The microbial cell extract helps decrease earthy/beany off flavours of plant-based fibres.
   The microbial cell extract shows excellent synergy with pea proteins and can replace pea fibre.
   The microbial cell extract shows good synergies with citrus fibres, forming textures similar to meat
   The microbial cell extract and bean meal blends result in good textures with a neutral sensory profile.
e. Other blends
   Adding the microbial cell extract to soy protein isolate (SPI) reduces brittleness and dryness when compared to SPI and SPI with gluten.
   Blends of SPI with gluten and microbial cell extract further improved the juiciness of the resulting extrudate.
   A blend of red lentils and microbial cell extract resulted in excellent textures that were reported as juicy, not brittle and not rubbery.
   A blend of 1:1 gluten:microbial cell extract results in a better, more juicy and less brittle texture in comparison to a 1:1.2 blend.
f. Adding the microbial cell extract to SPI and SPC reduces the toughness and rubbery behaviour of the extrudate.
g. The microbial cell extract provided an umami taste in extruded meat analogues.

### Example 14

Extruded samples containing the microbial cell extracts prepared as described in example 1 can be hydrated in a solution containing at least ˜1% of organic acids, including but not limited to acetic acid, for at least 5 minutes, which results in a texture with a more visually appealing colour.

### Example 15

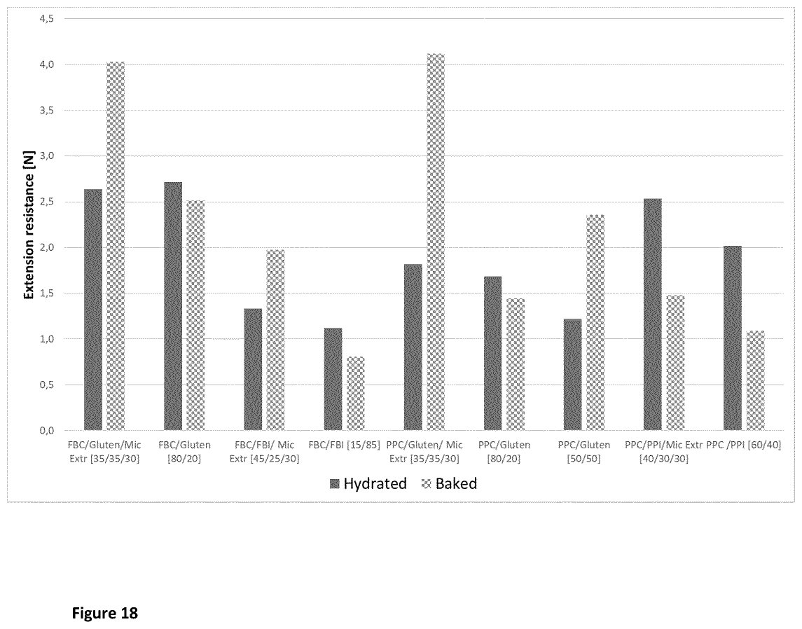

Microbial extracts are prepared in accordance with example 1, and the microbial cell extract heavy phase was dried using mild drying. The resulting powder was blended with with commercially available ingredients, fava bean protein isolate (FBI), fava bean protein concentrate (FBC), gluten, pea protein concentrate (PPC), and pea protein isolate (PPI). The resulting blends were then subjected to high moisture extrusion in a tween screw extruder at 135° C. and the resulting textures were then subjected to hydration as well as hydration plus baking in a pan for 5 minutes. After this, samples of the textures were taken and subjected to extension analysis in a texture analyser. Extension resistance was recorded up to 50% of the original length and the maximum extension resistance was chosen as output parameter. The resulting extension resistance was recorded and compared as shown in FIG. 18.

### Example 16

Extrusion trials were conducted in a tween screw extruder, performing high moisture extrusion at a temperature of 126° C. Samples containing the microbial cell extract heavy phase (mic exctr) as described in example 1, and yeast (food grade, active) biomass without treatment, were blended with commercial plant-based ingredients, pea protein isolate (PPI), fava bean protein isolate (FBI) as shown in table 2 below. The resulting extrudates were hydrated and the results are also summarised below.

TABLE 2
Qualitative assessment of extrudates prepared with the microbial
extract compared to extrudates prepare with intact whole yeast.

| | | Extrudates | | |
|---|---|---|---|---|
| Ingredients | Blend (wt %) | texture | | Hydration |
| PPI/Gluten/Mic | 27.5/27.5/45 | elastic | fibrous | fast |
| Extr | | | | |
| PPI/Gluten/Mic | 27.5/27.5/45 | elastic | fibrous | Fast |
| Extr | | | | |
| PPI/Gluten/yeast | 27.5/27.5/45 | brittle | flaky | medium |
| FPI/Gluten/Mic | 27.5/27.5/45 | elastic | Fibrous | fast |
| Extr | | | | |
| FPI/Gluten/yeast | 27.5/27.5/45 | brittle | flaky | Medium |

### Example 17

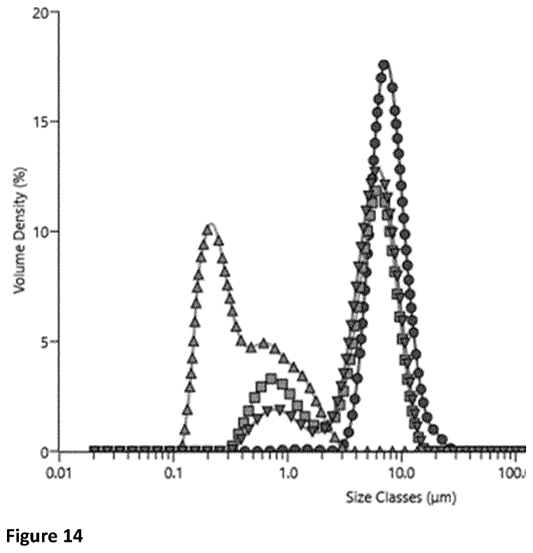

In an example demonstrating enrichment, a microbial biomass with a D50˜7.49 μm (depicted by the circles in FIG. 14) is disintegrated into a suspension with a bimodal distribution, represented by the squares in FIG. 14, with a D50 of ˜4.43 μm. The disintegrated biomass is subsequently separated into an extract enriched in small fragments, represented by the triangles with a D50˜0.35 μm, and a fraction enriched in large fragments, represented by the inverted triangles (FIG. 14) with a D50˜5.41 μm. Therefore, the light phase will be enriched in small fragments of sizes in the range 0.1-3 μm (D50<0.5 μm). Accordingly, the heavy phase will be enriched in large fragments of sizes>0.3 μm (D50>0.5 μm).

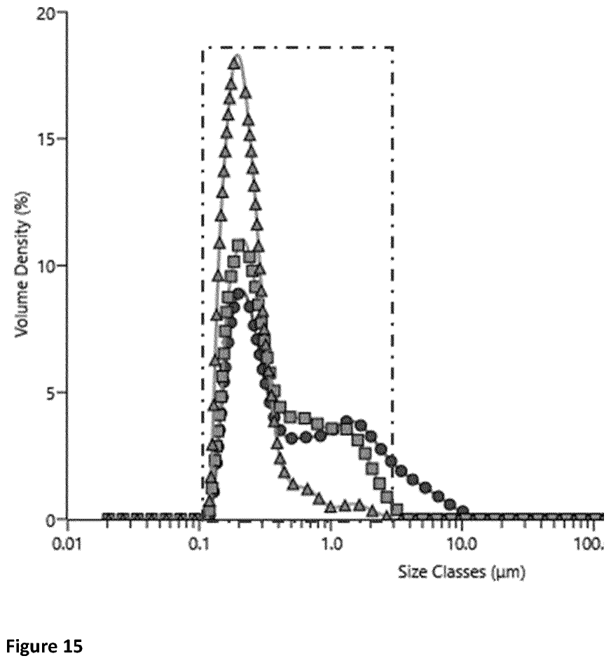

We have shown that there is a range of centrifugal forces that result in optimal functionality of the fraction enriched in small fragments. FIG. 15 shows a medium centrifugal force leads to superior enrichment of the fragments in the range of 0.1-3 μm, represented by the squares. Strong centrifugal forces (triangles) lead to a psd in the range of 0.1-0.4 μm, while low centrifugal forces result in a psd with fragments spanning to 10 μm (circles). The desired enrichment of small fragments is represented by the dashed square. If centrifugal forces are used which are too high or too low, this leads to a different psd and surprisingly worse functional performance, highlighting that obtaining a psd as defined by the present invention is critical.

Using different centrifugal forces affects the dry matter content of the light and heavy phase. Samples were produced according to the method of the invention as described above, but separated using mild and high centrifugal forces. Following separation these samples were subjected to the oven method known in the art, wherein the samples are kept at 100° C. in an oven until they are at a constant weight. The results of this are shown in table 3. A significant difference was observed in the dry matter content when different centrifugal forces were used. When using a mild centrifugal force of 4000×g for 15 minutes there was additional dry matter in the light phase, due to there being more small particles that remain in suspension. This is an advantage of using a mild centrifugal force to achieve separation.

TABLE 3
Dry matter contents of the light and heavy phase
after centrifugal separation of disrupted biomass
using high and mild centrifugal forces.

| | Dry |
|---|---|
| | Weight |
| | % |
| High Centrifugal | |
| force: 20000 xg | |
| 15 min | |
| Light phase | 3.9% |
| Heavy phase | 17.2% |
| Mild Centrifugal | |
| force: 4000 xg, | |
| 15 min | |
| Light phase = FESF | 6.1% |
| Heavy phase FELF | 13.5% |

In previous publications that have reported producing microbial protein concentrates and protein isolates from a soluble fraction after cell lysis and centrifugation, centrifugal forces in the range of 10000-30000×g are required to produce such soluble fractions and remove all insoluble compounds and particles. Traditionally, soluble fractions with a higher purity have been associated with high functionality and superior performance. However, unexpectedly the inventors have found that selectively enriching a fraction with small fragments resulted in superior functionalities as described above.

### Example 18

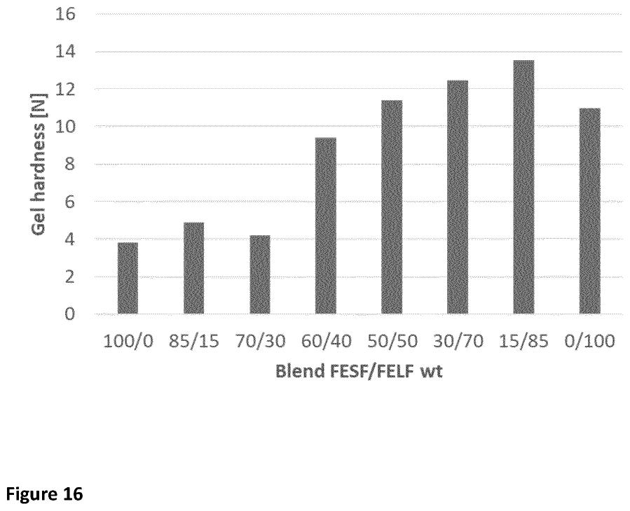

FIG. 16 shows several recombination ratios of the fraction enriched in small fragments and the fraction enriched in large fragments. The fractions were dried using spray drying recombined in a variety of ratios according to weight and assessed in terms of gelation hardness.

Gelation hardness was measured after heat-set gelation in a water bath (15% DW suspension, heated at 90° C. for 30 min, followed by cooling at room temperature for 20 minutes and measuring hardness using a Texture analyzer Lloyd TA-Plus).

FIG. 16 shows optimal gelation hardness was achieved with a ratio of 15:85 of the EESF to the EELF. For reference, in this example the approximate ratio of both fractions prior to recombination was 40:60 which yielded a gelation hardness of ˜8N.

It was unexpected that there was a ratio existing at which there was synergy between both fractions resulting in superior functionality, in this example, gelation hardness.

### Example 19

A microbial cell extract was prepared according to the method described in PCT/EP2021/075137 (WO2022/058287). In summary, said microbial cell extract is produced by i) providing an aqueous suspension comprising microbial cells; ii) subjecting said suspension to mechanical cell disintegration, to obtain an aqueous suspension comprising disintegrated microbial cells; and iii) separating the suspension to provide an extract enriched in small cell fragments (“light phase”), and an extract enriched in large cell fragments (“heavy phase”). It is noted that optionally at least a portion of each extract may be recombined, to provide a recombined microbial cell product.

In this example the aqueous suspension comprising microbial cells comprises biomass of Methylobacterium spp at ˜100 g/L adjusted to ˜pH 9 with NaOH and subjected to cell disintegration via bead milling using 0.3 mm Zirconium beads, with a 65% filling rate, agitation speeds of 2039 rpm, and a temperature of ˜20° C.

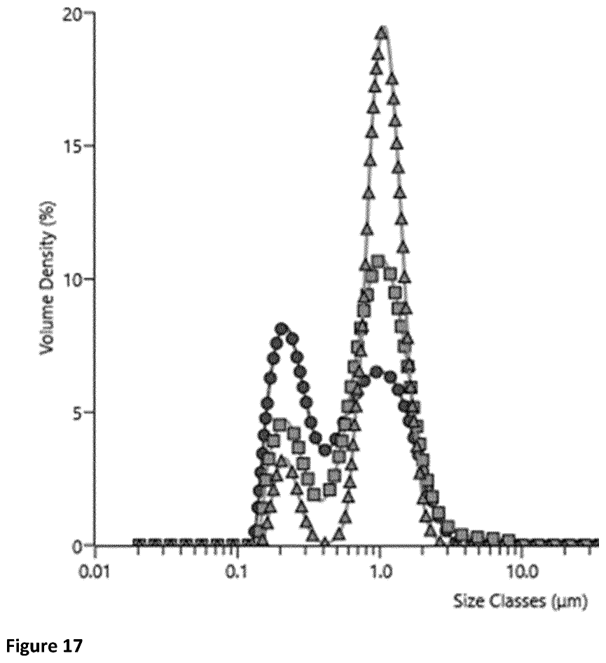

The resulting disintegrated biomass particle size distribution is shown in FIG. 17, and represented by triangles, here it can be seen that a D50˜1.03 μm was achieved.

After the disintegration step, the resulting microbial suspension was subjected to centrifugation using a batch centrifuge at 4000×g for 15 minutes at 15° C. This results in the formation of a light phase and a heavy phase of the microbial suspension (also referred to as an extract enriched in small cell fragments, and an extract enriched in large cell fragments, respectively).

The particle size distribution and particle sizes of the light and heavy fractions resulting from separation by centrifugation are shown in FIG. 17 where the squares represent the heavy fraction and the circles represent the light fraction. The light fraction had a D50 of ˜0.57 μm. Meanwhile, the heavy fraction had a D50˜0.9 μm.

The resulting fractions were then analysed according to their functional properties as shown in table 4 below. Here it is shown that the oil holding capacity and gelation performance were substantially improved in both the EESF and EELF compared to the disintegrated biomass.

TABLE 4
Functional properties of disintegrated Methylobacterium
spp and the resulting EESF and EELF

| Functional | Disintegrated | | |
|---|---|---|---|
| Property | Biomass | EESF | EELF |
| WHC [g/g] | 4.71 | 4.75 | 3.85 |
| OHC [g/g] | 2.23 | 3.15 | 4.61 |
| Gel | No, viscous | Yes | Yes |
| | paste | | |
| G′max[Pa] | 1.19 × 10⁵ | 1.17 × 10⁶ | 8.58 × 10⁵ |

1. A method of preparing a microbial cell extract, the method comprising:
a) providing a microbial biomass in an aqueous alkaline suspension, at pH 7-11;
b) mechanically disintegrating the microbial biomass at a temperature below 35 deg C. using a non-denaturing process, such that the disintegrated biomass consists of a population of soluble compounds and suspended fragments characterized by a bimodal distribution and a particle size distribution (psd) at an average of around D50<4.5 μm;
c) subjecting the disintegrated biomass to a solid-liquid separation process, to separate the disintegrated biomass into an extract rich in small fragments and an extract rich in large fragments, wherein the extract rich in small fragments consists of a population of soluble compounds and suspended fragments characterized by a bimodal distribution and a psd of D50 around 0.5 um or lower; and the extract rich in large fragments consists of a population of soluble compounds and suspended fragments having a psd of D50>0.5 um.

2. The method of claim 1, further comprising the step of:
d) recombining a portion of the extract rich in small fragments and a portion of the extract rich in large fragments to provide a microbial extract, preferably at a ratio of at least 65:35 large:small.

3. The method of claim 1, wherein the microbial biomass comprises microalgae, yeast, bacteria or fungi; preferably wherein the biomass comprises an organism selected from Saccharomyces, Pichia, Tetraselmis, Chlorella, Arthrospira, Fusarium, and Lactobacillus.

4. The method of claim 1, wherein the microbial biomass is prepared in an aqueous alkaline suspension at a concentration of 5 to 15% dry weight (DW).

5. The method of claim 1, wherein the mechanical disintegration step is carried out by bead milling, or homogenization.

6. The method of claim 1, wherein the disintegration step is carried out at a pH in the range of 7-11, and at a temperature in the range of 10-30° C.

7. The method of claim 1, wherein disintegration is carried out to obtain a population of soluble compounds and suspended fragments characterized by a bimodal distribution and a particle size distribution (psd) of D50<4.5 μm.

8. (canceled)

9. The method of claim 1, wherein the microbial cell extract has a protein content between 30-60%.

10. The method of claim 1, wherein the microbial cell extract has a total dietary fiber content of >20%.

11. The method of claim 1, wherein the microbial cell extract has an RNA content<5%

12. The method of claim 1, wherein the microbial cell extract has a fat content<10%

13-14. (canceled)

15. The method of claim 1, further comprising subjecting the microbial cell extract to membrane filtration.

16. The method of claim 1, further comprising drying the microbial cell extract to a powder with a moisture content<8%.

17. The method of claim 1, further comprising subjecting the microbial cell extract to thermochemical transformation.

18-19. (canceled)

20. The method of claim 1, further comprising spinning, electrospinning, heating, or 3d printing the microbial cell extract.

21. (canceled)

22. The method of claim 1, further comprising combining the microbial cell extract with one or more protein and/or fibre rich food ingredients, preferably a food ingredient derived from soy, pea, fava bean, lentil, wheat, bran, rice, oat, barley or citrus.

23. A method for preparing a substitute food composition, the method comprising:
a) providing a microbial biomass in an aqueous alkaline suspension, at pH 7-11;
b) mechanically disintegrating the microbial biomass at a temperature below 35 deg C. using a non-denaturing process, such that the disintegrated biomass consists of a population of soluble compounds and suspended fragments characterized by a bimodal distribution and a particle size distribution (psd) at an average of around D50<4.5 μm;
c) subjecting the disintegrated biomass to a solid-liquid separation process, to separate the disintegrated biomass into an extract rich in small fragments and an extract rich in large fragments, wherein the extract rich in small fragments consists of a population of soluble compounds and suspended fragments characterized by a bimodal distribution and a psd of D50 around 0.5 um or lower; and the extract rich in large fragments consists of a population of soluble compounds and suspended fragments having a psd of D50>0.5 um
d) optionally recombining a portion of the extract rich in small fragments and a portion of the extract rich in large fragments to provide a recombined microbial extract;
e) combining one or more of the extracts with one or more protein and/or fibre rich food ingredients; and
e) extruding the combined ingredients to provide a substitute food composition.

24. The method of claim 23, further comprising the step of blending the extruded product of step (e) with a portion of the microbial extract rich in small fragments.

25. A recombined microbial extract, the extract comprising a protein content between 30-60%, a total dietary fiber content of >20%, an RNA content<5%, and a fat content<10%.

26. The recombined microbial extract according to claim 25, wherein the extract has a water holding capacity of at least twice its own dry weight.

27. The recombined microbial extract according to claim 25, wherein the extract has an oil holding capacity of at least 1.6 times its own dry weight.

28. A substitute food product comprising the microbial extract of claim 25.

29-33. (canceled)
